(19) United States
(12) Patent Application Publication
(10) Pub. No.: US 2022/0047645 A1
(43) Pub. Date: Feb. 17, 2022

(54) METHOD FOR TREATING CARDIAC CONDITIONS WITH PLACENTA-DERVIVED COMPOSITIONS

(71) Applicant: Musculoskeletal Transplant Foundation, Edison, NJ (US)

(72) Inventors: Zain Khalpey, Oro Valley, AZ (US); Marc Long, Monmouth Junction, NJ (US); Pamela G. Hitscherich, Nutley, NJ (US)

(21) Appl. No.: 17/398,173

(22) Filed: Aug. 10, 2021

Related U.S. Application Data
(60) Provisional application No. 63/064,251, filed on Aug. 11, 2020.

(51) Int. Cl. A61K 35/50; A61K 9/00; A61K 9/70; A61P 41/00; A61P 9/00

(52) U.S. Cl. CPC A61K 35/50; A61K 9/0024; A61P 9/00; A61P 41/00; A61K 9/7007

(57) ABSTRACT

Provided herein are compositions and methods for treating heart disease. In particular, provided herein are constructs, devices, and systems, each comprising one or more placenta-derived compositions, and their use in treating disorders involving aberrant cardiac rhythms and promoting repair of damaged cardiac tissue. The method of treatment comprises applying one or more constructs on the surface of all or a portion of the heart and/or adjacent tissues, during or after surgical heart treatment.

### CROSS-REFERENCE TO RELATED APPLICATIONS

The present application claims the benefit of U.S. Provisional Application No. 63/064,251, filed Aug. 11, 2020, the entire disclosure of which is incorporated by reference herein.

### FIELD OF THE DISCLOSURE

The present invention relates to therapeutic compositions and constructs comprising those compositions, and methods for treating heart disease. In particular, provided herein are constructs, compositions, and systems comprising placenta-derived compositions, and methods for their use in treating cardiac disorders.

### BACKGROUND OF THE DISCLOSURE

Every year, more than 500,000 US patients undergo coronary artery bypass graft surgery (CABG) to treat obstructive coronary artery disease. The most common complication of CABG is new onset postoperative atrial fibrillation (NOPAF), which occurs in up to 35% of CABG patients and is most likely underestimated. NOPAF instigates ventricular arrhythmias, reduces cardiac output, causes renal failure and worsens postoperative mortality while increasing postoperative length of stay, pharmacological intervention, resource utilization, and readmission rate. It is believed that pro-inflammatory mediators from the surgical trauma most likely provoke NOPAF and may confer a prothrombotic state by promoting endothelial damage/dysfunction and platelet activation. Contributing factors secondary to surgery include excessive adrenergic stimulation, autonomic imbalance, neuro-humeral abnormalities, stretch induced phenomenon and type of surgical procedure used.

Although it is clear that trauma during the perioperative period triggers NOPAF, implementing a preventive or prophylactic strategy is complicated by adverse effects of pharmacotherapies.

Transmyocardial revascularization (TMR) is a surgical procedure that offers symptomatic relief to cardiac patients who have regions of ischemia. The procedure reduces the chest pain or discomfort of coronary heart disease, also known as angina pectoris. Angina often occurs when the heart muscle needs more blood than it is getting, often due to physical exertion. This procedure can be used as an adjunct to CABG or to improve the quality of life for some cardiac patients who might not otherwise be suited for treatment.

Additional compositions and methods for treating and preventing postoperative arrhythmias and other cardiac conditions are needed.

### SUMMARY OF THE DISCLOSURE

Methods are provided for treating or reducing a cardiac disorder in a subject having a heart, which comprise contacting the heart or a portion thereof with at least one construct comprising one or more placenta-derived compositions. In some embodiments, the cardiac disorder is one or more of: cardiac arrhythmias, scar tissue on the heart or a portion thereof.

Methods are provided for treating or reducing new onset postoperative atrial fibrillation (NOPAF) in a subject having a heart subjected to a surgical procedure, which comprise contacting the heart or a portion thereof with at least one construct comprising one or more placenta-derived compositions. The type of surgical procedure is not limited, so that the constructs and methods of use for treating NOPAF may be employed beneficially with (i.e., before, during, or after) any medical procedures performed to treat the heart and/or cardiac conditions in a subject.

Methods are provided for treating or reducing scar tissue on a heart or portion thereof, which comprise contacting the scar tissue with at least one construct comprising one or more placenta-derived compositions.

In some embodiments, contacting the heart or a portion thereof comprises placing the at least one construct on a surface of the heart or a portion thereof. In some embodiments, one or more of the constructs placed on the heart may be sheets, layers, particles, coatings, etc. In some embodiments, contacting the heart or a portion thereof comprises injecting at least one construct (such as by using a syringe or cannula) onto the surface of the heart, such as to form a layer or coating on the heart. In some embodiments, the method for treating the heart comprises forming one or more coatings or layers on the surface of the heart, or proximate thereto, wherein the coatings or layers may include more than one construct, more than one material (i.e., a different therapeutic or non-therapeutic substance), and combinations thereof. In some embodiments, contacting the heart or a portion thereof comprises placing the at least one construct on a surface of a tissue or tissues adjacent to the heart or a portion thereof, during a surgery or a procedure for treating cardiac muscle, where the adjacent tissue is in contact with the heart in a natural anatomy (ex. pericardium flap). In some embodiments, the at least one construct comprises two or more constructs and the method comprises placing each of the constructs in contact with the heart or a portion thereof, and each construct may, independently, be adjacent, overlapping, or not in contact with one or more of the other constructs. In some embodiments, the surgical procedure is a coronary artery bypass graft surgery (CABG) or a heart valve repair/replacement and contacting the heart or a portion thereof comprises placing the at least one construct on a surface of the heart or a portion thereof during the CABG or heart valve repair/replacement.

The placenta-derived compositions include one or more placental membrane components such as, without limitation, an amnion membrane, a chorion membrane, a portion thereof, or a combination thereof. In some embodiments, each of the placental membrane components has a form selected, without limitation, from: a sheet, a disc, a piece, a fragment, a particulate, a powder (e.g., a fine particulate), a three-dimensional shape, a coating, a layer, a film, an elongated element, and combinations thereof. In some embodiments, the one or more placental membrane components further comprise native endogenous cells, which may be viable or not.

In some embodiments, the placenta-derived composition comprises an amnion membrane sheet. In some embodiments, the placenta-derived composition comprises a chorion membrane sheet. In some embodiments, the placenta-derived composition comprises an umbilical cord sheet. In some embodiments, the placenta-derived composition comprises at least two or more of: an amnion membrane sheet, a chorion membrane sheet, and an umbilical cord sheet. In some such embodiments, one or more of the amnion membrane sheet, chorion membrane sheet, and umbilical cord sheet is dried or lyophilized. In some embodiments, the placenta-derived composition comprises a lyophilized amnion membrane sheet. In some embodiments, the placenta-derived composition comprises an amnion membrane sheet and a chorion membrane sheet, both of which are lyophilized.

In some embodiments, the placenta-derived composition comprises an amnion membrane sheet comprising viable native endogenous cells. In some embodiments, the placenta-derived composition comprises a chorion membrane sheet comprising viable native endogenous cells. In some embodiments, the placenta-derived composition comprises an umbilical cord sheet comprising viable native endogenous cells. In some embodiments, the placenta-derived composition comprises at least two of: an amnion membrane sheet comprising viable native endogenous cells, a chorion membrane sheet comprising viable native endogenous cells, and an umbilical cord sheet comprising viable native endogenous cells.

In some embodiments, the placenta-derived compositions include particulates from one or more placental components such as, without limitation, an amnion particulate or particulates, a chorion particulate or particulates, an umbilical cord particulate or particulates, or a combination thereof.

In some embodiments, the placenta-derived compositions are combined with an autologous preparation derived from the patient's own blood, such as a platelet-rich plasma, with or without the retention of autologous leukocytes.

### BRIEF DESCRIPTION OF THE DRAWINGS

The present invention will be further explained with reference to the attached drawings, wherein like structures are referred to by like numerals and/or letters throughout the several views. The drawings shown are not necessarily to scale, with emphasis instead generally being placed upon illustrating the principles of the present invention.

FIGS. 1A-1B are schematic diagrams showing an exemplary surgical site including a human heart, first with the pericardium open, exposing the heart and showing relative placement of three constructs (FIG. 1A), and then with the pericardium closed, covering the heart and showing the post-operative positions of the three constructs (FIG. 1B);

FIG. 2 shows surgical application of a placenta-derived graft (AMNIONBAND®) to an anterior left ventricle of a subject;

FIG. 3 shows another view of the placenta-derived graft (AMNIONBAND®) applied to an anterior left ventricle of the subject shown in FIG. 2;

FIG. 4 shows another view of the placenta-derived graft (AMNIONBAND®) applied over right atrium of the subject shown in FIG. 2; and

FIG. 5 shows another view of the placenta-derived graft (AMNIONBAND®) applied over diaphragm, juxtaposed to abut the inferior right ventricle of the subject shown in FIG. 2.

### DETAILED DESCRIPTION OF THE DISCLOSURE

### Definitions

As used herein, the term “subject” refers to individuals (e.g., human, animal, or other organism) to be treated by the methods or compositions of the present invention. Subjects include, but are not limited to, human and non-human animals, including mammals (e.g., human, non-human primates, rodents, ovines, bovines, ruminants, lagomorphs, porcines, caprines, equines, canines, felines, etc.), reptiles, and birds.

The terms “application,” “applying,” “placement,” “placing,” “administration,” “administering,” “implantation,” and “implanting,” as used herein in connection with the methods of using the placenta-derived compositions described and contemplated herein, are essentially synonymous and mean placement, with or without fixation (e.g., suture, adhesive, staple or other affixing means), of one or more such compositions at least partially in physical contact, directly or indirectly, with cardiac muscle, the heart, or components thereof of a subject (e.g., pericardium, epicardium, myocardium, endocardium, valve, aorta, vena cava, atrium, ventricle, etc.). While several exemplary embodiments of the methods for using the placenta-derived compositions are provided hereinbelow, the methods described and contemplated herein are not limited to those embodiments, but rather, any and all embodiments involving application, placement, or administration of the compositions which cause or result in physical contact of one or more placenta-derived compositions with cardiac muscle, the heart or components thereof, of a subject are included and covered within such methods.

As used herein, the term “native endogenous cells” means cells that are naturally occurring (endogenous) to a particular tissue type and were resident in a particular tissue sample of that type after recovery from a donor and remain resident in that tissue sample after any processing or manipulation (native). For example, amnion membrane typically contains one or more cells of several types such as, but not limited to, mesenchymal stem cells (MSCs) and fibroblasts, both before and after recovery from a donor and processing. After a sample of the amnion membrane is recovered, separated, subjected to size reduction, cleaning, disinfection, drying, and other techniques any MSCs and/or fibroblast cells that were present in that sample prior to processing and still remain in that tissue after said processing, are characterized as native endogenous cells.

As used herein, the term “therapeutic agent,” refers to compositions that decrease the infectivity, morbidity, or onset of mortality in a subject (e.g., a subject with a heart condition). As used herein, therapeutic agents encompass agents used prophylactically. The constructs described and contemplated herein are, themselves, considered therapeutic agents.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

As used herein, the terms “placenta” and “placental” mean any one or more components or parts of a mammalian placenta including, without limitation, amnion, chorion, amniochorion, allantois, amniotic fluid, umbilical cord, and Wharton's jelly. Sources of placental components are not particularly limited and include, without limitation, human, non-human primate, porcine, equine, bovine, and combinations thereof.

As described in further detail hereinbelow, it has been demonstrated that human placental membrane allograft/patch placement during coronary artery bypass graft surgery (CABG) reduces the incidence of new onset postoperative atrial fibrillation (NOPAF). Without wishing to be limited by theory, it is believed that this benefit is achieved by suppression of pro-inflammatory mediators by human amniotic membrane, and possibly other placental components. Human placental membrane is immunologically privileged and includes basic components necessary for tissue generation and anti-inflammation. It is known, for example, that human amniotic membrane tissue retains anti-microbial, anti-adhesion and anti-fibrotic capabilities, which may also be mechanisms for observed reduction in NOPAF using human amniotic membrane.

Accordingly, methods are provided herein for treating or reducing a cardiac disorder in a subject having a heart, which comprise contacting the heart or a portion thereof with at least one construct comprising one or more placenta-derived compositions. In some embodiments, the cardiac disorder is one or more of: cardiac arrhythmias, scar tissue on the heart or a portion thereof.

Additionally, methods are provided herein for treating or reducing new onset postoperative atrial fibrillation (NOPAF) in a subject having a heart subjected to a surgical procedure, which comprise contacting the heart or a portion thereof with at least one construct comprising one or more placenta-derived compositions.

Methods are also provided herein for treating or reducing scar tissue on a heart or portion thereof, which comprise contacting the scar tissue with at least one construct comprising one or more placenta-derived compositions.

In some embodiments, contacting the heart or a portion thereof comprises placing at least one construct on a surface of the heart or a portion thereof. In some embodiments, contacting the heart or a portion thereof comprises delivering a placental allograft in particulate form, in suspension, as a dry powder, with a carrier, or another suitable formulation, onto the at least one construct on the heart or a portion thereof. In some embodiments, the at least one construct comprises two or more constructs and the method comprises placing each of the constructs in contact with the heart or a portion thereof, and each construct may, independently, be adjacent, overlapping, or not in contact with one or more of the other constructs. In some embodiments, the surgical procedure is a coronary artery bypass graft surgery (CABG) and contacting the heart or a portion thereof comprises placing the at least one construct on a surface of the heart or a portion thereof during the CABG.

The placenta-derived compositions include one or more placental membrane components such as, without limitation, an amnion membrane, a chorion membrane, a portion thereof, or a combination thereof. Such embodiments are not limited to particular sources of the placental membrane components. In some embodiments, the placental membrane components are, for example, without limitation, of human origin, or adult or fetal porcine origin. Among other characteristics, placental membrane components suitable for use in the methods described and contemplated herein have no more than about 0.05 endotoxin units per milliliter (EU/mL; wherein one EU equals approximately 0.1 to 0.2 no endotoxin/mL) or about 20 EU/composition endotoxin levels, as determined by limulus amebocyte lysate (LAL) testing. As generally understood by persons of ordinary skill in the relevant art, endotoxins are components of the outer layer of the outer cell membrane of Gram negative bacteria and often cause fever in subjects exposed to or in contact with them.

In some embodiments, each of the placental membrane components are, independently of one another, whole, discontinuous, perforated or meshed, particulate, and/or solubilized. Furthermore, each of the placental membrane components may, for example without limitation have a form selected from: a sheet, a piece, a fragment, a particulate, a powder (e.g., a fine particulate), a three-dimensional shape, a coating, a layer, a film, an elongated element, and multiples and combinations thereof. In some embodiments, the one or more placental membrane components further comprise native endogenous cells, which may be viable or not. Such cells include, without limitation, mesenchymal stem cells (MSCs), fibroblast cells, stromal cells, and epithelial cells.

In some embodiments, the placenta-derived composition comprises an amnion membrane sheet. In some embodiments, the placenta-derived composition comprises a chorion membrane sheet. In some embodiments, the placenta-derived composition comprises an amnion membrane sheet and a chorion membrane sheet. In some embodiments, the placenta-derived composition comprises a lyophilized amnion membrane sheet. In some embodiments, the placenta-derived composition comprises an amnion membrane sheet and a chorion membrane sheet, both of which are dehydrated or lyophilized. Without wishing to be limited by theory, it is believed that contacting a construct comprising two or more layers of amnion membrane, chorion membrane, or at least one of each, with a heart or portion thereof postoperatively provided improved anti-inflammatory effect and more effective reduction of NOPAF, than constructs comprising only one such amnion or chorion membrane.

In some embodiments, the placenta-derived composition comprises an amnion membrane sheet comprising viable native endogenous cells. In some embodiments, the placenta-derived composition comprises an amnion membrane sheet comprising viable native endogenous cells, and a chorion membrane sheet comprising viable native endogenous cells. In some embodiments, the placenta-derived composition comprises: an amnion membrane sheet, a chorion membrane sheet, and an umbilical cord comprising viable, one or more of which comprises native endogenous cells.

In some embodiments, the placenta-derived composition is cryopreserved, refrigerated, or stored at ambient temperature.

As will be readily recognized by persons of ordinary skill in the relevant art, in addition to one or more placenta-derived compositions, the construct may further comprise one or more additional components. Such additional components may include polymer materials, therapeutic agents (e.g., pharmaceutical compounds, growth factors, viable cells, devitalized cells or cell components, other tissue-derived materials, corticosteroids, antibodies, cytokines, proteins, morphogenic proteins, etc.), anti-microbial agents, anti-infective agents, anti-inflammatories, steroids and cortico-steroids, anti-adhesion agents, analgesics, scaffold or other support or carrier material, and the like.

For example, in some embodiments, the construct may further comprise polymer materials, which may be acellular or not. Such polymer materials may be natural polymers, synthetic polymers, or combinations thereof. The association or manner of combination of the polymer materials with the placental membrane components is not particularly limited. For example, without limitation, the polymer material may be complexed, conjugated, encapsulated, absorbed, adsorbed, or admixed, with one or more placental membrane component to form the construct. In some embodiments, the placental membrane components are grown to between 50-90% confluency on a polymer material. In some embodiments, the resulting construct has a size and shape appropriate for application onto myocardium tissue of a mammalian (e.g., human) subject. In some embodiments, the construct may further comprise one or more other naturally-occurring or -derived materials, including but not limited to: hyaluronan, chondroitin sulfate, glucosamine, collagen, silk, and polypeptides.

In some embodiments, in addition to one or more placental membrane components, the constructs may further comprise additional components such as one or more proteins and/or growth factors (e.g., including but not limited to, vascular endothelial growth factor, platelet-derived growth factor, transforming growth factor, fibrinogen, collagen alpha-3(VI), fibronectin, collagen alpha-1(XII) chain, vimentin, collagen alpha-1(VII) chain, transforming growth factor-beta-induced protein, prelamin, laminin subunit alpha-3, laminin subunit alpha-5, desmoplakin, decorin, Pentraxin-related protein PTX3, Fibrillin-1, Annexin A2, Lumican, mucin-16, or epiplakin).

In some embodiments, the construct further comprises a rigid or semi rigid frame. In some embodiments, the frame being disposable or implantable and resorbable. In some embodiments, the construct is an allograft configured for engagement with mammalian tissue (e.g., heart tissue, such as, for example, epicardial surface tissue, endocardial surface tissue, myocardial tissue, left atrial tissue, right atrial tissue, valvular tissue, purkinje network, pericardial tissue, vascular, perivascular (AV-fistula) anastomoses, and/or peritubular tissue).

In some embodiments, additional components combined with the placenta-derived compositions to form the constructs include one or more of: a stromal vascular fraction, a cellularized atrial appendage, a de-cellularized atrial appendage, adherent material, cormatrix, platelet-rich plasma, induced pluripotent stem cells, neonatal cardio-ventricular myocytes, embryonic stem cells, cardiac stem cells, mesenchymal stem cells (MSCs), fibroblast cells myoblast cells, adult bone marrow-derived cells, mesenchymal cells, endothelial progenitor cells, umbilical cord blood cells, T regulatory cells, M2-like macrophages, skeletal muscle satellite cells, muscle myoblast, C2C12 muscle myoblast cell line, fibroblasts, or combinations thereof.

In some embodiments, in addition to one or more placental membrane components, the constructs may further comprise additional components such as one or more anti-inflammatory therapeutic agents. In some embodiments, the one or more anti-inflammatory therapeutic agents may, for example, include corticosteroids (Dexamethasone), glucocorticoids (prednisolone), anti-thrombotic, anti-arrhythmic (amiodorone), beta-blockers, ace inhibitors, angiotensin-receptor blockers, anti-oxidants (resveratrol; natural and synthetic), statins, anti-inflammatory nanoparticles (antigenic), antibodies (immunotherapy), anti-inflammatory cytokines, or gene therapy (plasmids, AVVs).

In some embodiments, the construct is configured for direct and targeted application onto a desired tissue region. In some embodiments, the construct is configured (i.e., has a form as described above which is suitable) for paint on, roll on or spray application. In some embodiments, the construct is in a powder based form, an emulsified form, a fluid based form, an aerosol based form, or a gel based form.

Further embodiments provide a method for preventing and/or treating a subject suffering or at risk of suffering from a disorder characterized by aberrant cardiac function, comprising applying one or more constructs described herein to the heart of a subject, wherein the amount of each of the one or more constructs is an amount effective to treat the disorder. In some embodiments, the aberrant cardiac function is one or more of new onset postoperative atrial fibrillation, atrial arrhythmia, ventricular arrhythmia, atrial fibrillation, atrial flutter, epicardial inflammation, myocardial inflammation, aberrant contractile function, aberrant cardiac conduction, aberrant cardiac rhythms, myocardial infarction, congestive heart failure, or heart failure with preserved ejection fraction. In some embodiments, the disorder characterized by aberrant cardiac function is new onset postoperative atrial fibrillation following coronary artery bypass graft surgery. In some embodiments, the construct is applied to the heart of a subject during a coronary artery bypass graft surgery. In some embodiments, the duration of time to apply the construct to the heart of a subject is within 2-10 minutes. In some embodiments, the construct is applied to at the site of vessel graft over the left atria extending to the base of the heart during the coronary artery bypass graft surgery. In some embodiments 2 or more (e.g., 2, 3, 4, 5, 6, 7, or more) constructs are provided. The constructs described herein find use in a variety of applications. For example, in some embodiments, the construct is applied to the heart of a subject following valve replacement and/or repair over the left atria extending to the base of the heart. In some embodiments, the subject's left ventricle has therein mechanical circulatory support devices, wherein the construct is applied at the site of mechanical circulatory support devices in the left ventricle. In some embodiments, the construct is applied at the site of vessel graft over the left atria extending to the base of the heart following coronary artery bypass graft surgery. In some embodiments, the construct is applied to the heart of a subject following valve replacement and/or repair over the left atria extending to the base of the heart. In some embodiments, the construct is applied to the heart of a subject at the pericardial space using synthetic or biological materials. In some embodiments, the one or more constructs are applied to (e.g., contacted with) the heart of a subject by minimally invasive techniques, for example, by endoscopic and/or trocar techniques.

Additional embodiments provide a method of treating postoperative atrial fibrillation in a subject (e.g., mammalian (e.g., human) subject), comprising applying one or more constructs described herein to the heart tissue of a subject. Without intending to be limited by theory, it is believed that applying of the one or more constructs to the heart tissue of the subject facilitates modulation of cardiac function and/or modulation of cardiac related electrical activity. In some embodiments, the applying of the one or more constructs to the heart tissue of the subject is injecting or spraying the one or more constructs into the heart tissue of the subject.

In some embodiments, the heart, portion of the heart, or heart tissue with which the one or more construct are contacted include, without limitation, myocardial tissue, epicardial surface tissue, or endocardial surface tissue. In some embodiments, the post-operative atrial fibrillation results from myocardial infarction treatment.

In some embodiments, the one or more constructs are frozen, pulverized, and mixed prior to application.

In certain embodiments, the present invention provides kits comprising (a) a plurality of constructs as described herein; and (b) instructions on applying the constructs to a tissue region of a subject.

Further embodiments provide a method for improving cardiovascular remodeling and revascularization in a subject, comprising: administering a combination of a construct described herein and transmyocardial revascularization to the heart of said subject. In such embodiments, at least one of the one or more constructs contacted with the heart or portion thereof further comprises stem cells. This embodiment provides a method for remodeling and reducing scars on heart tissue.

Provided herein are constructs comprising one or more placenta-derived compositions such as human amniotic membrane and/or human chorionic membrane for use in preventing heart arrhythmias and promoting repair of scarred regions of the heart.

Human placental membrane is a highly abundant and readily available tissue. This amniotic tissue has considerable advantageous characteristics to be considered as an attractive material in the field of regenerative medicine. It has low immunogenicity, anti-inflammatory properties and their cells can be isolated without the sacrifice of human embryos. Since it is discarded post-partum it may be useful for regenerative medicine and cell therapy. Human amniotic membrane contains two cell types, from different embryological origins, which display some characteristic properties of stem cells. Human amnion epithelial cells (hAECs) are derived from the embryonic ectoderm, while human amnion mesenchymal stromal cells (hAMSCs) are derived from the embryonic mesoderm.

### I. Constructs

Provided herein is a construct comprising one or more placenta-derived compositions, with or without additional components such as those described in further detail hereinbelow. Each of the one or more placenta-derived compositions may, independently of one another, comprise one or more placental components and each of which may have the shape or form of: a sheet, a piece, a fragment, a particulate, a powder (e.g., a fine particulate), a three-dimensional shape, a coating, a layer, a film, an elongated element, and combinations thereof.

The size and shape of a construct according to an embodiment of the present invention vary depending on the surgery the construct is to be used. In some embodiments, the construct is 1 to 10 cm in width and length (e.g., 1, 2, 3, 4, 5, 6, 7, 8, 9, 10 cm or other size), and the width and length may be equal or different from one another. The construct is not limited to particular shapes. According to embodiments of the present invention, a construct having the general form of a sheet may further be shaped as a quadrilateral, square, rectangular, oval, round, donut shape, etc. According to embodiments of the present invention, the construct can optionally have one or more slits that allow easy application to the surgical site, or easy access of veins/vessels or other tissues at the application site. The split can be made by various methods, such as by cutting the construct at the desirable position while the construct is still wet.

The present disclosure is not limited to a particular formulation of the placenta-derived compositions or constructs comprising the same. Examples include but are not limited to, fluid based form, emulsified form, powder based form, aerosol based form, or gel based form. In some embodiments, the one or more constructs are frozen, pulverized, and mixed prior to application.

In one embodiment of the present invention, one or more corners of the construct are rounded or flattened to prevent the corners from catching during implantation. In view of the present disclosure, any method known to those skilled in the art can be used to make the corners of the construct round or flatten.

In some embodiments, the construct is configured for injectable application and/or spray application. In some embodiments, the construct is in a powder based form, an emulsified form, a fluid based form, an aerosol based form, or a gel based form.

The present disclosure is not limited to a particular source or method of obtaining amnion membrane. Prior to expulsion or recovery from a donor subject, amnion membrane generally has two surfaces: (1) an outer surface in contact with chorion tissue; and (2) an inner surface in contact with amniotic fluid. Likewise, prior to expulsion or recovery from a donor subject, chorion membrane also generally has two surfaces: (1) an outer surface that is contact with maternal cells; and (2) an inner surface that is in contact with amnion tissue.

Amnion has a complete lack of surface antigens, thus does not induce an immune response when implanted into a ‘foreign’ body or subject, which is in contrast to most other allograft implants. Amnion also markedly suppresses the expression of the pro-inflammatory cytokines, IL-1α and IL-1β. Amnion also down-regulates TGF-β and its receptor expression by fibroblasts leading to the ability to modulate the healing of a wound by promoting tissue reconstruction. Furthermore, amnion and chorion contain antimicrobial compounds with broad spectrum activity against bacteria, fungi, protozoa, and viruses for reduced risk of post-operative infection.

Amnion and chorion membranes, as well as umbilical cord tissue can be prepared, separately or together, from birth tissue procured from a pregnant female mammal (human or non-human). Sterile techniques and procedures should be used as much as practically possible in tissue handling, e.g., during tissue procurement, banking, transfer, etc., to prevent contamination of the collected tissues by exogenous pathogens.

Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4° to 28° C., for example, in an ice bucket.

According to an embodiment of the invention, shortly after its expulsion after birth, a suitable human placenta is placed in a sterile bag, which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The amnion and chorion membranes are separated from the other tissues of the placenta and then separated from each other using methods known in the art and in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, each of the isolated amnion and chorion is treated, independently or together, with one or more antibiotics, such as penicillin and/or streptomycin, prior to cryopreservation.

Amnion and chorion extracellular matrix components include heavy-chain hyaluronic acids, growth factors, fibronectin, and collagen and are preserved with the membrane during cryopreservation and other isolation methods.

The isolated amnion and chorion membranes are tough, transparent, nerve-free and nonvascular sheets. They can be dried or lyophilized using various methods. For example, these membranes can be dried (together or separately) over a sterile mesh, for example, by being placed on a sterile nitrocellulose filter paper and air dried for more than 50 minutes in a sterile environment. They can also be dried or lyophilized over another form of supporting material, which would facilitate the subsequent manipulation of the membranes, such as sterilizing, sizing, cataloging, and shipping. Umbilical cord is another component of the placenta and, after recovery and removal of the umbilical vein and arteries, may be processed similarly to the amnion and chorion membranes as described above.

In some embodiments, the construct can be made by drying amnion and/or chorion membranes and acellular materials into the required shape over a frame, such as an implantable and resorbable frame, e.g., polymer mesh frame, or a disposable or stainless steel frame. Preferably, the frame is rigid or semi rigid. The frame can be any of the shapes suitable for the surgery, e.g., triangle, rectangle, quadrilateral, oval, donut, circle, semicircle, etc.

In an embodiment of the present invention, when a disposable frame is used, the dried tissue retains the shape of the frame when removed from the frame or could be packaged and sterilized with or without the disposable frame. The disposable frame can be removed and discarded prior to the use of the tissue. The disposable frame can be longer than the tissue for ease of handling and removal, or ease of application to the incision or surgical site.

In another embodiment of the present invention, an implantable and resorbable frame is used. Such a frame could be a mesh or a solid frame with several holes throughout.

The human amnion and/or chorion membranes, are bonded to the frame by various methods in view of the present disclosure, such as, drying the tissue on the frame, using a resorbable adhesive, keeping the tissue wet and laying it on the frame, or freezing the tissue on the frame. Examples include, but are not limited to, complexed, conjugated, encapsulated, absorbed, adsorbed, or admixed. In some embodiments, the placental membrane components are grown to between 50-90% confluency on the construct.

In some embodiments, the construct is freeze-dried and milled or pulverized into particulate form. In some embodiments, the construct comprises a disposable or implantable and resorbable frame (rigid or semi-rigid), polymeric materials (natural, synthetic, or combinations thereof), and placental membrane components, wherein the placental membrane components may further comprise amnion membrane cells, chorion membrane cells, or combinations thereof. In such embodiments, the construct may be produced by associating the polymeric materials and placental membrane components with the frame such that the placental membrane components grow to between 50-90% confluency on the frame; freeze-drying the construct, and milling or pulverizing the construct to produce a construct in particulate form.

In some embodiments, the constructs further comprise one or more additional components, such as without limitation, growth enhancing agents, morphogenic proteins, small molecule compounds, pharmaceutical agents, anti-microbial agents, anti-inflammatory agent, agents that prevent scarring, adhesions and tethering of internal tissue at or near the surgery site, analgesics, etc., to further improve the performance and reduce the complications of abdominal surgeries. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing.

In some embodiments, constructs further comprise one or more additional components selected from, but not limited to antibacterial compounds, including bactericidal and bacteriostatic compounds, antibiotics (e.g., adriamycin, erythromycin, gentimycin, penicillin, tobramycin or vancomycin), antifungal compounds, anti-inflammatories, antiparasitic compounds, antiviral compounds, enzymes, enzyme inhibitors, glycoproteins, growth factors (e.g. lymphokines, cytokines), hormones, steroids, glucocorticosteroids, immunomodulators, immunoglobulins, minerals, neuroleptics, proteins, peptides, lipoproteins, tumoricidal compounds, tumorstatic compounds, toxins and vitamins (e.g., Vitamin A, Vitamin E, Vitamin B, Vitamin C, Vitamin D, or derivatives thereof). It is also envisioned that selected fragments, portions, derivatives, or analogues of some or all of the above may be used.

In some embodiments, the methods described and contemplated herein comprise providing a kit comprising a construct for use in treating or preventing cardiac disease and, optionally, instructions on how to use the construct. Any of the constructs described herein according to embodiments of the present invention can be included in the kit.

### II. Methods of Use

The placental-derived compositions and constructs comprising one or more placental-derived compositions described herein find use in any number of uses related to treating, preventing, or modulating a variety of cardiac conditions. The constructs further find use in research and screening application. Exemplary uses are described herein.

In some embodiments, the constructs are used to prevent new onset postoperative atrial fibrillation following coronary artery bypass graft surgery. Atrial Fibrillation after cardiac surgery occurs in approximately one third of patients and is associated with increased rate of readmissions, complications and death. Inflammation and oxidative stress may play a key role in the pathophysiology of new onset postoperative atrial fibrillation (NOPAF).

In some embodiments related to preventing NOPAF, the construct is applied to the heart of a subject during a coronary artery bypass graft surgery. In some embodiments, the construct is applied to at the site of vessel graft over the left atria extending to the base of the heart during the coronary artery bypass graft surgery.

In some embodiments, the constructs are applied to the heart of a subject following valve replacement and/or repair over the left atria extending to the base of the heart.

In some embodiments, the construct is applied at the site of mechanical circulatory support devices in the left ventricle.

In some embodiments, the construct is applied to the heart of a subject following valve replacement and/or repair over the left atria extending to the base of the heart.

In some embodiments, a construct comprising one or more placenta-derived compositions, each of which have the form of a sheet, layer or patch, is applied to the heart of a subject, such as on the pericardial surface of the heart, using synthetic or biological materials. For example, in some embodiments, three such constructs are applied to the surface of, and/or proximate to, the pericardium of the heart of a subject. For example, without limitation, in an exemplary embodiment where three such constructs are used, a first construct is clipped (or otherwise affixed with an affixing device) to the right heart pericardium, a second construct is clipped (or otherwise affixed with an affixing device) to the diaphragmatic pericardium to abut the inferior right ventricle and a third construct is placed topically over the anterior right/left ventricle and the pericardium is placed over the topically-placed third construct it to keep it in place.

In another exemplary embodiment where three such constructs are used, a first construct is clipped (e.g., with a blue load medium (3-5 mm) metal clip) or another suitable metal clipping device or means) to the pericardial edge in order to approximate placement over the right atrium, and a second construct is clipped to the pericardial edge to approximate placement over the anterior left ventricle, once the pericardium is closed; and a third construct is placed at the base of the heart and clipped, or otherwise affixed there, to cover the inferior right ventricle. Where more than one construct is applied to the heart, each may or may not be affixed thereto, and may be affixed by the same device or means or not. The affixing or clipping device and its material of construction are not particularly limited and may be any biocompatible device suitable for effectively affixing the construct to the pericardial edge and suitable for remaining in the subject post-surgery.

In some embodiments, at least two constructs, each of which comprises one or more placenta-derived compositions, are applied to the heart of a subject. In some embodiments, three such constructs are applied to the heart. In embodiments wherein two or more such constructs are applied to the heart, the constructs may or may not contact or overlap with one another, or not. In some embodiments, each placenta-derived composition (and therefore each construct comprising same), independently, may have dimensions of length×width of, for example without limitation, about 5×6 cm, or about 5×7 cm, or about 6×6 cm, or about 7×7 cm, or about 6×8 cm, or about 6×10 cm, or about 8×10 cm, or about 10×10 cm.

In some embodiments, wherein more than one construct is applied to the heart of a subject, each construct has a surface area and the sum total of the surface areas of all applied constructs should advantageously be from at least about 30 to about 150 square centimeters (cm²). In some embodiments, for example without limitation the total of the surface areas of the applied constructs is from about 10 to about 170 cm², such as from about 10 to about 120 cm², or from about 30 to about 120 cm², or from about 40 to about 110 cm², or from about 40 to about 100 cm², or from about 50 to about 100 cm², or from about 20 to about 80 cm². For example, without limitation, in an embodiment wherein three constructs, each of which comprises one or more placenta-derived compositions and has a surface area, the total of the surface areas of the constructs may be at least about 90 cm².

In some embodiments, two constructs are applied to the heart of a subject and each construct has dimensions of about 7×7 cm. In some embodiments, three constructs are applied to the heart of a subject and each construct has dimensions of about 5×6 cm. It is noted that, in any embodiment where more than one construct is applied to the heart, the constructs need not be the same size as one another, nor must they be of the same composition as one another. Furthermore, without wishing to be limited by theory, it is believed that, while the use of two constructs is effective for treating the heart (e.g., preventing or minimizing NOPAF, etc.), applying three constructs, each of which is in accordance with the presently described and contemplated invention, to the heart will provide a further improvement in efficacy of the treatment.

In some embodiments the constructs are placed in defined areas of the heart; at minimum on the right atrium, anterior right ventricle, inferior right ventricle, anterior right & left ventricle.

In some embodiments, the constructs are applied to (e.g., contact with) the heart (or adjacent tissues) of a subject by minimally invasive techniques, for example, by endoscopic, robotic and/or trocar techniques.

In some embodiments, constructs are administered via injection or spraying onto the surface of the heart, or the surface of adjacent tissues.

The constructs described herein find use in application to any number of different heart tissues. In some embodiments, the heart tissue is myocardial tissue, epicardial surface tissue, or endocardial surface tissue.

In some embodiments, the constructs find use in treatments to promote revascularization and remodeling of scarred heart tissue. Cardiac scar initially consists of necrotic cardiomyocytes and tissue, which are replaced with granulation tissue consisting of fibrin, fibronectin, laminin, GAGs and other matrix proteins. Over time, myofibroblasts infiltrate the tissue and remodel to a stiff and more fibrous scar based mostly upon collagen. One conventional treatment for regions of the scarred heart is to perforate the tissue to provoke revascularization and remodeling of the tissue. Such revascularization can promote survival of cardiomyocytes and permit cardiac stem cells to repopulate the area. While earlier versions of this therapy utilized needle-like arrays to puncture the myocardium the current conventional therapy is to use a CO₂ laser to puncture the tissue. Although called transmyocardial revascularization or TMR, there is little evidence that the channels produced by the laser actually permit or localize blood vessels. Nevertheless, there is some improvement in cardiac output after treatment. Similarly, injection of stem cells of various origins has been another approach to improve or restore cardiac function in scarred and hypertrophied hearts. In some embodiments, the constructs described herein are used in combination with TMR (e.g., as a patch at the site of TMR).

In some embodiments, one or more of the therapies described herein is provided in combination with a gene therapy treatment (e.g., using an adeno associated viral vector (AAV) or other vector). Examples of adenoviral vectors and methods for gene transfer are described in published PCT application Nos. WO 00/12738 and WO 00/09675, and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

For example, in some embodiments, gene therapy is locally delivered to the heart (e.g., through the coronary sinus of the heart) at time of amnion membrane component therapy or at a different time. In some embodiments, gene therapy approaches utilize single or multiple plasmid synthetic human genes which don't create fusion proteins (e.g., which won't minimize antibodies). Examples of genes for use in gene therapy include, but are not limited to, s-100, SDF or VEGF.

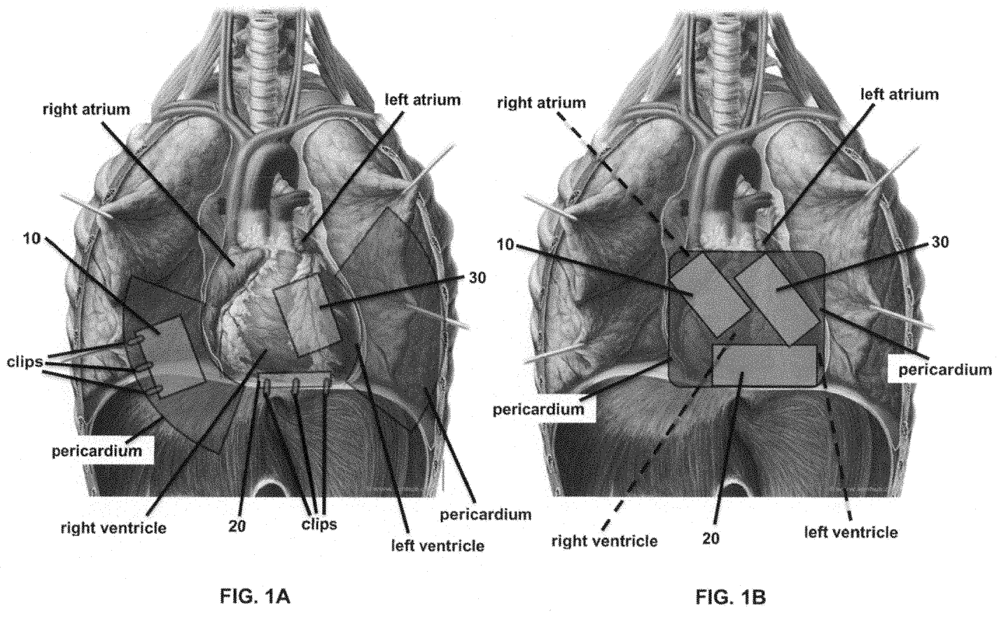

The following description is an exemplary embodiment of the presently described and contemplated method and applies to the application of a construct comprising one or more placenta-derived compositions onto the surface of, or proximate to, the heart of a subject. Reference is made to FIGS. 1A and 1B, which provide schematic diagrams showing an exemplary surgical site including a human heart, first with the pericardium open, exposing the heart and showing relative placement of three constructs 10, 20, 30 (FIG. 1A), and then with the pericardium closed, covering the heart and showing the post-operative positions of the three constructs 10, 20, 30 (FIG. 1B).

It is noted that while the description below concerns using three constructs 10, 20, 30, each comprising an amnion-derived composition and a chorion-derived composition, both of which are derived from human placenta and both of which have been lyophilized (hereinafter referred to as a “dehydrated amnion/chorion allograft, hereinafter “dHACA”), the following method is provided as an exemplary embodiment and, therefore, is applicable to the use of any one or more constructs comprising any one or more placenta-derived compositions, which may be the same or different from one another, as otherwise described and contemplated above.

### Preoperative Preparation

1. Aseptic processing of placental tissue or tissues
Endotoxin assay testing (optional)
   a. Done at source of placenta derived compositions/construct
2. Gram stain testing
   a. If going into an infected wound
      i. Dip and swirl three times the membrane in heparinized normal saline in a culture cup
      ii. Send solution for gram positive/negative staining & culture
      iii. This should be done prior to placement in the mediastinum
      iv. This can be done on the back table of the surgical field
3. Three 5 cm×6 cm dHACA constructs 10, 20, 30 (see FIGS. 1A-1B), each of which is in a sheet or patch shape (e.g., a total 90 cm² topical dose of human allograft amnion/chorion membrane) should be opened on the surgical field

### Intra-Operative Steps

1. Heparinized Saline
   a. Prior to placement, place 2 ml heparinized normal saline over one dry 5 cm×6 cm dHACA construct (10), while in opened packet
2. Right pericardium placement (Right atrium), from the surgeon's side (right of patient's chest)
   a. Use Russian forceps ×2 and pick edges of first wet dHACA construct (10)
   b. Present Lower half of right atrium vertically
   c. Approximate first wet dHACA construct (10) lengthwise to cut edge of pericardial membrane
   d. Place 3 blue clips over first wet dHACA construct (10) and clip to cut pericardial edge (see FIG. 1A)
3. Diaphragm placement (Right Ventricular placement), from the surgeon's side (right of patient's chest)
   a. Use Russian forceps ×1 and pick the middle/center of second wet dHACA construct (20)
   b. Use the left hand to gently push back the right ventricular edge covering the diaphragmatic surface
   c. Surgical first assistant can help approximate the edges and clip the second wet dHACA construct (20) to the diaphragmatic surface
   d. Three blue clips (3-5 mm metal clips) should be used to approximate the medial, middle and lateral clips to the cut diaphragmatic pericardial edge.
   e. Make sure the diaphragmatic pericardial edge is dry and has good hemostasis.
   f. See below for instructions on how to place left mediastinal chest tube and ventricular wire placement
4. Anterior ventricular placement (Anterior left ventricular placement), from the surgeon's side (right of patient's chest)
   a. The surgical first assistant will need to assist by lifting pericardial stitches on the cut pericardial edge of the left pericardium
   b. The anterior ventricle (closer to the left anterior descending artery territory should be identified)
   c. A Russian forceps ×2 should be used to pick up the edges of the third wet dHACA construct (30)
   d. This should be placed over the lower half of the left anterior descending artery territory, which may include the anterior right ventricle (⅓) and anterior (½) of the left ventricle.
   e. The native pericardium is approximated over the placed third dHACA construct (30) and pericardium approximated over the dHACA construct to secure it in place (see below).
5. Pericardial approximation, from the surgeon's side (right of patient's chest)
   a. Use a 1.0 silk pericardial stitch and approximate the left and right pericardium over the aorta, just over the aortic cannulation site to approximate just below the aorto-innominate junction. Tie this down with 4 knots
   b. Use a 1.0 silk pericardial stitch and approximate the left and right pericardium over the root of the aorta. Tie this down with 4 knots
   c. After each of the right pericardial (first), diaphragmatic (second), and anterior ventricular (third) dHACA constructs 10, 20, 30 have been placed, use a 1.0 silk pericardial stitch to approximate the lower right cut pericardial edge, then to the mid diaphragmatic cut edge and continue with the same stitch to go through the cut edge of the left cut pericardial edge. Approximate this “v” shaped stitch to bring the edges together. Tie this down with 4 knots.
6. Pacing wires, from the surgeon's side (right of patient's chest)
   a. Atrial wires
      i. Two bipolar wires for atrial wires can be placed on the atrial appendage
      ii. The wires should be placed in the right mediastinal gutter (pericardial reflection and inferior vena cava)
      iii. Wires should be placed BEFORE placement of the right pericardial (first) dHACA construct (10)
      iv. Make sure that the first dHACA construct (10) has maximal exposure to the right atrial tissue
   b. Ventricular wire
      i. The right inferior ventricular bipolar wire (X1) should be placed in the mid, right inferior ventricular belly
      ii. The ventricular wire should be placed BEFORE placement of the diaphragmatic pericardial (second) dHACA construct (20)
      iii. The wire should exit in the mid-clavicular line above the diaphragm and every attempt should be made to avoid coming close to the diaphragmatic placed (second) dHACA construct (20)
7. Chest tube placement, from the surgeon's side (right of patient's chest)
   a. 24 French pericardial Blake mediastinal chest tubes ×2 (right and left mediastinal chest tubes)
   b. Place right mediastinal chest tube after right pericardial (first) dHACA construct (10) has been clipped.
   c. Place this in the right mediastinal gutter, just above the inferior vena caval—right atrial junction, below the first wet dHACA construct (10) and direct the tip of the cut mediastinal Blake chest tube close to the superior vena caval—right atrial junction
   d. After clipping the diaphragmatic pericardial (second) dHACA construct (20), take a Russian forceps in the right hand and take the cut tip of the left mediastinal Blake; use the left hand and place the index finger over the right ventricular wire (on the diaphragmatic right ventricular surface) and depress the ventricle superiorly, and place the tip of the mediastinal chest tube below the apex of the left ventricle.
   e. −20 cm H₂O of suction should be placed on the pleural evacuation device of the mediastinal chest tubes after approximation of the pericardial edges, prior to closure of the sternum.
8. Sternal wire placement and sternal closure
   a. A lap pad should be placed over the approximated pericardium prior to closing the sternum
   b. 8 surgical steel wires are used (or closure per preference of the surgeon's normal method)
   c. Removal of lap pad after good hemostasis and final approximation of sternum
   d. Monitoring of hemodynamics (arterial line, and/or Swan-Ganz catheter placement if necessary for cardiac surgical indication; it is not necessary to have a Swan-Ganz catheter for the placement of human allograft membrane) at time of sternal closure
   e. Deep, superficial fascia and skin are closed in layers per normal surgical fashion

### EXAMPLES

The following examples are provided to demonstrate and further illustrate certain embodiments of the present disclosure and are not to be construed as limiting the scope thereof.

### Example 1

Implantation of Human Placenta-Derived Composition Decreases New Onset Postoperative Atrial Fibrillation (NOPAF).

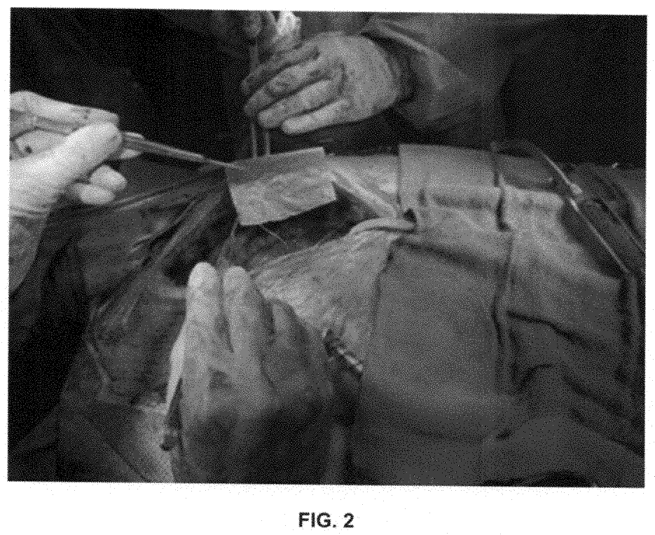

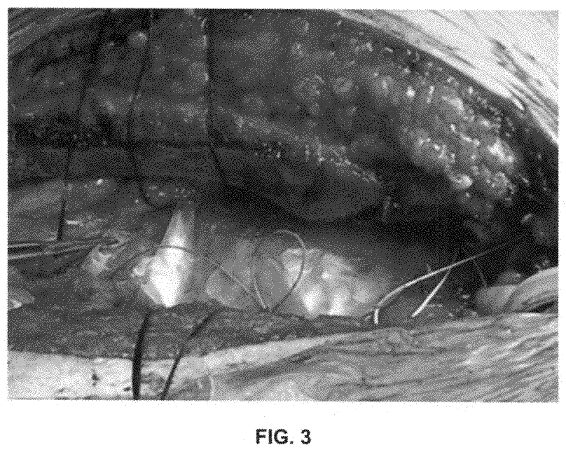

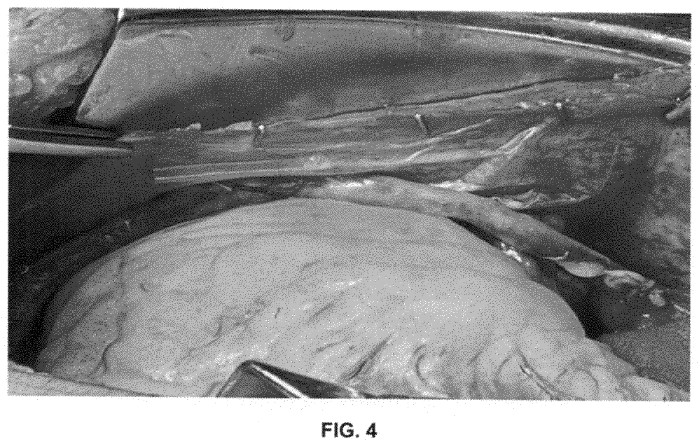

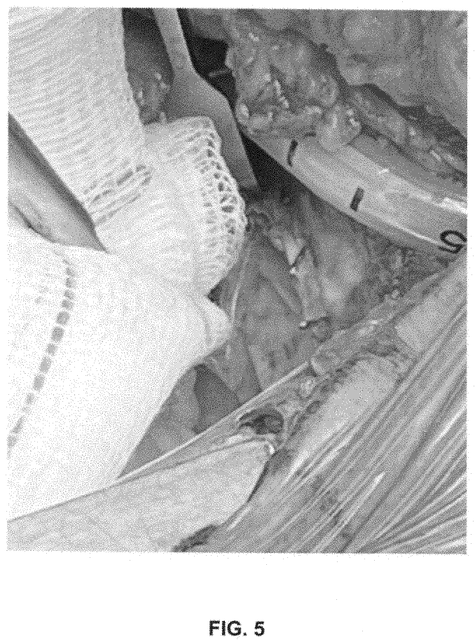

Patients undergoing coronary artery bypass (CABG) surgery were treated or not with a dehydrated human placental (amnion/chorion) allograft amnion/chorion membrane (“PAM”), i.e., treated (+PAM) or not (−PAM). More particularly, the PAM was a dHACA construct, composed of aseptically processed, lyophilized and laminated amnion and chorion membranes (commercially available in various sizes, under the tradename AMNIOBAND® Membrane, from Musculoskeletal Transplant Foundation of Edison, N.J., U.S.A., “MTF”). PAM allografts of size 5 cm×6 cm were used in this experiment. Patients were excluded from analysis if they presented with renal failure (Glomular Filtration Rate decrease of 50% and Creatinine increase of 0.3), heart failure (ejection fraction <35%), previous treatment of atrial fibrillation or it was determined by the physician that the patients required concurrent treatments (including but not limited to platelet rich plasma, transmyocardial revascularization, valve replacement). Most patient demographics were similar between groups and are summarized in Table 1 (Independent Samples T-test or nonparametric alternative or Fisher's Exact Test for dichotomous data). Thus far, 32 CABG(−PAM) patients and 14 CABG(+PAM) patients have been included in the study. Treatment for each of the 14 CABG(+PAM) patients involved placement of 3 PAM patches, with 2 clipped to the pericardium in order to approximate placement of one PAM patch over the anterior left ventricle (FIGS. 2 and 3) and another PAM patch over the right atrium (FIG. 4), and a third PAM patch placed at the base of the heart to cover the inferior right ventricle (FIG. 5). CABG(−PAM) patients did not receive any PAM placement. Of the patients who were treated with PAM, only 1 has developed NOPAF compared to 37.5% of CABG(−PAM) patients (Table 2).

TABLE 1
Patient Demographics.

| | CABG(−PAM) | CABG(+PAM) | |
|---|---|---|---|
| Variable | (mean ± STD) | (mean ± STD) | p value |
| Gender (female) | 37.50 | 14.28 | 0.043* |
| Age | 68.34 ± 8.78 | 64.21 ± 6.73 | 0.040* |
| BMI | 30.40 ± 5.41 | 30.07 ± 4.26 | 0.843 |
| MI (prior) | 34.38 | 42.86 | 0.585 |
| Pre-Op Ejection Fraction (%) | 54.69 ± 6.97 | 56.07 ± 7.79 | 0.471 |
| STS Score (%) | 1.19 ± 0.77 | 0.71 ± 0.64 | 0.018* |
| Vessels Involved | 2.62 ± 0.71 | 3.00 ± 0.63 | 0.116 |
| Cross-Clamp Time (min) | 60.31 ± 30.10 | 66.50 ± 19.98 | 0.747 |
| Bypass Time (min) | 93.50 ± 37.88 | 104.93 ± 31.33 | 0.229 |

TABLE 2
NOPAF Incidence

| Group | Total | −NOPAF | +NOPAF (%) | p value |
|---|---|---|---|---|
| CABG − PAM | 32 | 20 | 12 (37.50) | 0.022* |
| CABG + PAM | 14 | 13 | 1 (7.14) | |

### Example 2

Implantation of Human Placenta-Derived Composition Decreases New Onset Postoperative Atrial Fibrillation (NOPAF) in Patient with Heart Failure (HF) and Renal Failure (RF).

Patients undergoing coronary artery bypass (CABG) surgery who also had renal failure (Glomular Filtration Rate decrease of 50% and Creatinine increase of 0.3) or heart failure (ejection fraction <35%) were treated or not with PAM, i.e., treated (+PAM) or not (−PAM) as described in Example 1. Three PAM allografts, each of size 5 cm×6 cm, were used in this experiment and implanted in substantially the same positions as described in Example 1. Patients were excluded from analysis if they presented with previous treatment of atrial fibrillation or it was determined by the physician that the patients required concurrent treatments (including but not limited to platelet rich plasma, transmyocardial revascularization, valve replacement). Patient demographics summarized in Table 3 (if able Independent Samples T-test or nonparametric alternative or Fisher's Exact Test for dichotomous data). Both patients with HF and RF treated with PAM had lower incidence of NOPAF than those who were untreated (Table 4).

TABLE 3
Patient Demographics

| | Heart Failure Patients | | Renal Failure Patients | | |
|---|---|---|---|---|---|
| | CABG(−PAM) | CABG(+PAM) | CABG(−PAM) | CABG(+PAM) | |
| Variable | (mean ± STD) | (mean ± STD) | (mean ± STD) | (mean ± STD) | p value |
| Gender (female) | 50 | 60 | 8.33 | 33.33 | 0.193 |
| Age | 61.50 ± 3.30 | 64.60 ± 10.34 | 71.00 ± 7.37 | 64.33 ± 8.01 | 0.098 |
| BMI | 28.11 ± 3.19 | 27.34 ± 4.29 | 30.40 ± 3.96 | 29.79 ± 5.03 | 0.963 |
| MI (prior) | 50 | 40 | 33.33 | 50 | 0.496 |
| Pre-Op Ejection | 40.00 ± 0 | 35.00 ± 9.94 | 54.17 ± 5.58 | 50.00 ± 4.18 | 0.132 |
| Fraction WO | | | | | |
| STS Score (%) | 0.68 ± 0.15 | 1.11 ± 0.72 | 1.36 ± 0.88 | 1.73 ± 1.93 | 0.888 |
| Vessels Involved | 2 ± 0.96 | 2.80 ± 0.53 | 3.08 ± 0.71 | 1.67 ± 0.84 | 0.263 |
| Cross-Clamp | 44.00 ± 19.73 | 69.80 ± 23.01 | 77.33 ± 24.98 | 64.00 ± 27.91 | 0.326 |
| Time (min) | | | | | |
| Bypass Time | 71.50 ± 37.07 | 126.00 ± 48.19 | 114.00 ± 28.96 | 93.00 ± 26.58 | 0.164 |
| (min) | | | | | |

TABLE 4
NOPAF Incidence

| | Heart Failure Patients | | Renal Failure Patients | | |
|---|---|---|---|---|---|
| Group | −NOPAF | +NOPAF (%) | −NOPAF | +NOPAF (%) | p value |
| CABG − PAM | 1 | 1 (50) | 5 | 7 (58.33) | 0.738 |
| CABG + PAM | 4 | 1 (20) | 3 | 3 (50) | |

### Example 3

Treatment with PAM v Treatment with Platelet Rich Plasma (PRP)

Platelet rich plasma (PRP) treatment has been used to promote wound healing and regeneration and to reduce inflammation. Because of this, patients undergoing CABG procedure were either treated with PRP (+PRP) alone or in combination with PAM treatment (+PRP+PAM) as described in Table 5. PRP treatment involved isolation of the patient's blood, centrifuging first at 1250 G for 1-3 minutes followed by 1050 G for 6-9 minutes to obtain a highly concentrated PRP. Ten mL of PRP is then combined with 5 mL of CaCl/Throm bin mix, consisting of 5 mL of calcium chloride to 5000 units of thrombin. (For detailed description and specific preparation of the PRP used in these procedures, see section below—PRP Preparation.)

Prior to mixing the PRP with CaCl/Throm bin (above), the mixture was used with transmyocardial laser (TMR) and 0.5 ml of 2 ml of the red fraction (platelet rich plasma, PRP preparation) of the PRP mixture was then injected 0.5 cm around the center of the TMR lesion into the myocardium with the heart arrested. The TMR lesion was concentrated around a scar zone or where there is a poor surgical target (that is, sub millimeter vessel for revascularization). The operator would create 10× lesions; each created with an 8 watt burst of energy (Cryolife energy source, FDA approved device, USA). If no TMR was performed, the same volume of the red fraction of the PRP mixture was injected in the peripheral border and into the zone of the scar or poor surgical target zone.

PRP treatment alone did not offer any benefit to patients regarding NOPAF. PAM treatment in any form decreased the incidence of NOPAF while the combination of +PRP+PAM seemed to have a synergistic effect as demonstrated by no incidence of NOPAF with combination therapy.

TABLE 5
Treatment Permutation Details and Outcomes - Examples 1, 2 and 3

| | | Size | | | | +NOPAF |
|---|---|---|---|---|---|---|
| Treatment | N | (cm) | # | Placement | Secured | % |
| CABG | 64 | — | — | — | — | 43.75 |
| Only | | | | | | |
| CABG + | 5 | — | — | — | — | 60.00 |
| PRP | | | | | | |
| CABG + | 7 | 7 × 7 | 2 | 1) Right atrium | No | 14.28 |
| PAM | | | | 2) Anterior aspect of the pericardium | | |
| | 1 | 7 × 7 | 3 | 1) Right atrium | No | 0 |
| | | | | 2) Anterior aspect of the pericardium | | |
| | | | | 3) Anterior aspect of the pericardium | | |
| | 1 | 5 × 6 | 1 | Anterior aspect of the pericardium | No | 0 |
| | 3 | 5 × 6 | 2 | 1) Right atrium | No | 33.33 |
| | | | | 2) Anterior aspect of the pericardium | | |
| | 27 | 5 × 6 | 3 | 1) Right atrium | Yes, | 22.22 |
| | | | | 2) Anterior aspect of the pericardium | clips | |
| | | | | 3) Base of the pericardium | | |
| CABG + | 2 | 7 × 7 | 2 | 1) Right atrium | No | 0 |
| PRP + | | | | 2) Anterior aspect of the pericardium | | |
| PAM | | | | | | |
| | 2 | 5 × 6 | 2 | 1) Right atrium | No | 0 |
| | | | | 2) Anterior aspect of the pericardium | | |
| | | | | 1) Right atrium | | |
| | 7 | 5 × 6 | 3 | 2) Anterior aspect of the pericardium | Yes, | 0 |
| | | | | 3) Base of the pericardium | clips | |

Platelet Rich Plasma (PRP)—Description and Preparation

Platelet-rich plasma (PRP) is a fraction of blood plasma with a different platelet concentration as compared to human whole blood. The platelet content of PRP is typically 6×10¹¹ platelets/ml. PRP is obtained by repeatedly centrifuging and washing whole blood of humans at different centrifugal speeds, and mixing different concentrations of platelets and anticoagulants. PRP typically contains a lot of cytokines, for instance, EGF, TGF-β, PDGF, and IGF-1. As discussed and used herein, PRP means an autologous concentration of human platelets that is 3 to 5 times greater than physiologic concentration of thrombocytes in whole blood, where normal platelet count in healthy human individual typically ranges between 150000 and 350000 cell/μL of whole blood. Without wishing to be limited by theory, it is believed that when platelets arrive at a tissue site, such as damaged or injured tissue, the platelets signal locally present stem cells to activate. Once the stem cells activate, they recognize the damaged cells and turn (e.g., differentiate) into the types of cells needed to produce, reconstruct, and/or heal the type(s) of tissue at the tissue site. Thus, introduction or implantation of PRP during surgical procedures, such as but not limited to CABG, is believed to be beneficial as facilitating or enhancing healing.

Process of Preparing PRP

Device/Machine: Harvest Smart Prep 2, Used to prepare PRP/PPP from sample of patient blood.

Speeds:

1-3 minutes into the process 2500 RPM (G force: 1250)

6-9 minutes into the process 2300 RPM (G force: 1050)

Products/Supplies Used:

CaCl/Thrombin (smaller syringe)—5 ml of calcium chloride to 5000 units of thrombin.
Calcium is used to reverse the anticoagulant effects of ACDA used in 60 ml syringe with 2 ml of ACDA 58 ml of patient whole blood (ACDA=Anticoagulant Citrate Dextrose Solution, Solution A, which is used as an anticoagulant in extracorporeal processing with autologous PRP systems in production of PRP.)
Thrombin activates platelets
   Cytokines (proteins in cell signaling) increase with platelet concentration, having more growth factors. Stem cells are attracted to GF, which stimulate cell division.
      Growth Factors:
         PDGF, platelet derived growth factor: chemoattraction for WBC and stems cells,
         TGF-beta, transforming growth factor-beta: promotes mitosis and increases collagen type 1 production, and
         VEGF, vascular endothelial growth factor: stimulates angiogenesis (development of new blood vessels).

Ratio of PRP to CaCl/Thrombin (platelet gel) mix: 1:2
5 ml of CaCl: 10 ml of PRP
PPP—yellow cup: 25 ml of platelet poor plasma—less concentrated in platelets
PRP—red cup: 10 ml of platelet rich plasma—more concentrated in platelets

Procedure

The PRP was obtained from the blood of patients before centrifugation (58 ml of whole blood drawn with 2 ml of ACDA). After centrifugation (14-16 minutes using the Harvest Smart Prep 2) using a 60 ml cup provided in the PRP kit, and according to their different density gradients, the separation of blood components (red blood cells, PRP, and platelet-poor plasma [PPP]) occurred in the 60 ml cup.

25 ml of PPP was aspirated from the 60 ml cup, using a yellow syringe provided in the PRP kit. A buffy coat/PRP and red blood cells remained in the 60 ml cup. 10 ml or less of PRP is then aspirated from the 60 ml cup, alongside a coat of red blood cells (RBC) that was deposited in the bottom of the 60 ml cup. PRP, PPP and CaCl/Thrombin mix were then passed off separately to a sterile field for mixing at the appropriate time of the application.

### Example 4

PAM Reduces Infarct Size and Promotes Recovery of Function in Porcine MI Model.

Male domestic farm pigs (3-4 months old, acquired from S&S Farms, San Diego, Calif.) were randomly assigned to myocardial infarction (MI only) (n=4) or MI with PAM (MI(+PAM), n=3). The PAM was the same type of allograft as used in Example 1 above (i.e., commercially available as AMNIOBAND® Membrane from MTF of Edison, N.J.). Briefly, MI was induced via a percutaneous ischemia-reperfusion protocol by inflating a catheter guided angioplasty balloon in the left anterior descending coronary artery for 45 minutes to cause occlusion. After MI induction and successful reperfusion, MI(+PAM) group underwent a median hem i-sternotomy to expose the left ventricle where a single PAM was sutured to the infarcted zone. Transthoracic echocardiography was performed prior to, immediately after and 2 weeks after surgery before sacrifice. Ejection fraction decreased for both groups immediately after MI but was significantly recovered at 2 weeks post-MI in MI(+PAM) group compared to MI only (46.77±2.73 v. 35.81±4.47, respectively, p=0.014). Additionally, upon sacrifice, heart tissue sections were stained with 2,3,4-triphenyl tetrazolium chloride (TTC) to visualize the infarct. TTC staining demonstrated a significant reduction in infarct size in MI(+PAM) pigs compared to MI only (11.03% v 22.02%, respectively, p=0.039). Picrosirus red staining demonstrated a significant difference in collagen content between the infarct zone and the remote, healthy zone, in the MI only group suggesting fibrotic remodeling (p<0.01). However, in the MI(+PAM) group there was no significant difference in collagen content between the infarct and healthy zones, suggesting reduced damage and fibrosis (p=0.31). Sections were also stained for CD206, a marker of M2a anti-inflammatory macrophages, and MI(+PAM) infarct zone tissue demonstrated an increasing trend in M2a macrophage infiltration compared to MI infarct zone and healthy controls, suggesting immunomodulatory effects of PAM in the post-MI microenvironment.

### Example 5

Prospective Clinical Trial to Evaluate Safety and Efficacy of PAM Allografts in Patients Undergoing Cardiac Bypass Surgery.

Study Population: Up to 100 male or female (ages 60-80) adults undergoing elective, on-pump cardiac surgery requiring bypass; coronary artery bypass grafting (CABG) are recruited. After a subject has undergone their preoperative evaluation and has been determined to be eligible for the clinical trial, subjects will be entered into study design. The first phase is performed to test the safety of the use of PAM (i.e., the bilayer dHACA construct described and used in Examples 1 and 2 above, commercially available from MTF, of Edison, N.J.). The randomized portion of the study is conducted after a review of the safety data. The prospective randomized controlled study evaluates the efficacy of the PAM technology in reducing the incidence of NOPAF following open heart surgical procedures on subjects undergoing first-time, isolated coronary artery bypass grafting. Safety and efficacy of the procedure is assessed from the operative procedure through 30 days post-procedure. The first 8 subjects are assigned to receive PAM. If 2 or fewer patients experience adverse events (AEs) believed related to PAM, and with DSMB approval, the study proceeds to the randomization stage. Patients should not have received any other investigational therapies 30 days prior to enrollment or during study duration.

Intervention and Randomization: Subjects are randomized into either the treatment or sham group in a 1:1 ratio, using stratified block randomization, with blocks of size 4, 6, or 8. Subjects randomized to the PAM treatment will have three PAM allografts placed on the epicardium prior to closing, while sham group undergoes opening and closing of autologous pericardial sac without placement of PAM or any other graft. Sham treatment was chosen over “placebo” patch to minimize patient risk. After randomization, the subject undergoes routine CABG receiving three PAM allografts on the epicardium prior to closing if entered into the treatment group. A Reveal LINQ (Medtronic) insertable cardiac monitor is implanted to analyze rhythms in real-time (or any other device that provides a single or two lead electrocardiogram to confirm the presence or absence of atrial fibrillation). The device is placed under the skin in the chest during surgery and records heart rate, heart rate variability and abnormal rhythms up to 3 years.

Endpoints: The primary safety endpoint is a composite of procedure-related serious adverse events occurring within 30 days postoperative. Secondary safety endpoints include bypass graft failure, and procedure related events occurring within 30 days. The primary efficacy endpoint is NOPAF within 10 days postoperatively, or hospital discharge, whichever is sooner. Additional efficacy outcomes include measures of pericardial inflammation (T2-weighted MRI at day 3 post-op), inflammatory biomarkers including C-reactive protein, TNF-, IL-2, IL-6, and IL-8, atrial fibrillation burden, incidence of perioperative MI, incidence of tamponade, as well as metabolomics from blood samples pre- and post-operatively in the OR, and post-op day 3.

Statistical Analysis Plan: Logistic regression is used to evaluate the reduction in odds of NOPAF events associated with PAM treatment in an intent-to-treat analysis. The regression model includes terms for an intercept (placebo NOPAF rate), PAM treatment effect, sex, and age, which is strongly associated with NOPAF events. Bayesian inference using Markov chain Monte Carlo is used to evaluate PAM treatment effect. An informative prior centered at log(0.2/0.8) is used for placebo log odds of NOPAF, while a mildly informative prior centered at zero (no effect) is used for the PAM treatment effect. The placebo prior distribution is based on the STS data (Table 2), but is down-weighted to place 90% or prior probability between rates of 0.1 and 0.35, thus allowing greater uncertainty in the baseline rate. The trial is considered successful if the PAM treatment indicates a reduction in incidence of NOPAF of at least 50% compared with placebo, with greater than 90% posterior probability.

### Example 6

Cellular and Molecular Mechanisms of PAM Allograft.

PAM allografts are applied directly to the heart (epicardially, including ischemic area) after I/R injury.

A PAM (i.e., bilayer dHACA allograft described and used in Examples 1 and 2 above, commercially available as AMNIOBAND® Membrane from MTF of Edison, N.J.), and a viable (wet/not dehydrated) amnion allograft containing live cells, PAM(V) (available under the tradename AMNIOBAND® Viable from MTF, of Edison, N.J.), are assayed for evaluation. Pigs are randomly assigned to 1 of 3 groups: (1) control (MI only), (2) PAM, or (3) PAM(V). Pigs are subjected to 60 minutes of ischemia and sacrificed at 60 days post-MI. Blood is drawn at 2 hours following reperfusion and cardiac Troponin I (cTnI) is measured to validate the extent of cardiac damage. Non-infarct pigs are also included as allograft controls.

Experimental Methods for Determining Cardiac Remodeling.

Serial transthoracic echocardiography: Echocardiography is performed prior to MI surgery and again after 10 days, 30 and 60 days post-MI using LogicE Ultrasound. Two-dimensional M-mode echocardiographic images are obtained from the parasternal short-axis views at the level of the mid-ventricles. Cardiac chamber dimensions and the left ventricular wall thickness are measured. Ejection fraction (EF), left ventricular volume (LV Vol) left ventricular posterior wall thickness (LVPW) and internal dimension (LVID) are measured from the M-mode images. Data is analyzed using Vevo 2100 analytic software. Data is obtained in triplicate and averaged.

Histological analysis: Following the 60-day period of reperfusion, hearts are rapidly excised, sectioned and double-stained with Evans blue and triphenyltetrazolium chloride (TTC) to define infarct size, area at risk and area of necrosis, and prepared for histological and immunohistological analysis. For assessment of collagen, picrosirius stained tissue sections are captured with a polarized light microscope camera (Axio Imager M1, Zeiss, Oberkochen, Germany) to detect birefringence of collagen fibers. The images are quantified by a semiautomated imaging analysis program (AxioVision, Zeiss). A color threshold is defined in such a way to detect mature collagen. The area of birefringence is then normalized by the total area of interest (Chen H, Hwang H, McKee L A, Perez J N, Regan J A, Constantopoulos E, Lafleur B, and Konhilas J P. Temporal and morphological impact of pressure overload in transgenic FHC mice. Frontiers in physiology. 2013; 4(205); Danilo C A, Constantopoulos E, McKee L A, Chen H, Regan J A, Lipovka Y, Lahtinen S, Stenman L K, Nguyen T V, Doyle K P, et al. Bifidobacterium animalis subsp. lactis 420 mitigates the pathological impact of myocardial infarction in the mouse. Beneficial microbes. 2017; 8(2):257-69; Konhilas J P, Watson P A, Maass A, Boucek D M, Horn T, Stauffer B L, Luckey S W, Rosenberg P, and Leinwand L A. Exercise can prevent and reverse the severity of hypertrophic cardiomyopathy. Circ Res. 2006; 98(4):540-8; Perez J N, Chen H, Regan J A, Emert A, Constantopoulos E, Lynn M, and Konhilas J P. Effects of chemically induced ovarian failure on voluntary wheel-running exercise and cardiac adaptation in mice. Comparative medicine. 2013; 63(3):233-43).

In order to characterize ventricular remodeling, cardiac sections are evaluated with antibodies against: CD68, macrophage marker to measure of inflammation; pan-actin to identify myocytes; vimentin to identify nonepithelial mesenchymal cell populations such as fibroblasts; Pecam 1 as a marker for angiogenesis; and ALDH1A1 as a mesenchymal stem cell marker. For a global assessment of inflammation, sections are stained with Russell Movat pentachrome for differential staining of elastic fibers, nuclei, muscle, fibrinoid and collagen; Masson's trichrome to evaluate muscle, collagen fibers, fibrin, and erythrocytes, and Miller's elastic stain for elastic fibers, nuclei, muscle and collagen as described (Khalpey Z, Penick K, Constantopoulos E, Garcia J, Sweitzer N, Runyan R, and Konhilas J. Meeting abstract submitted to American Heart Association. 2014; Stelly M, and Stelly T C. Histology of CorMatrix bioscaffold 5 years after pericardial closure. The Annals of thoracic surgery. 2013; 96(5):e127-975; Zaidi A H, Nathan M, Emani S, Baird C, Del Nido P J, Gauvreau K, Harris M, Sanders S P, and Padera R F. Preliminary experience with porcine intestinal submucosa (CorMatrix) for valve reconstruction in congenital heart disease: Histologic evaluation of explanted valves. J Thorac Cardiovasc Surg. 2014). Sections are also stained with antibodies against c-kit for stem cells, von Willbrand factor for endothelial cells and neovascularization, telomerase for evidence of cells proliferation, and CD34 for dendritic cells, endothelial cells and hematopoietic progenitors, CD3, CD5 and CD8 for T cells and CD20 for B cells. Finally, at the interface of the necrotic and viable myocardium is a region of intense cellular activity termed the border zone (Driesen R B, Verheyen F K, Dijkstra P, Thone F, Cleutjens J P, Lenders M H, Ramaekers F C, and Borgers M. Structural remodelling of cardiomyocytes in the border zone of infarcted rabbit heart. Mol Cell Biochem. 2007; 302(1-2):225-32; Gottlieb G J, Kubo S H, and Alonso D R. Ultrastructural characterization of the border zone surrounding early experimental myocardial infarcts in dogs. Am J Pathol. 1981; 103(2):292-303). I/R injury is very localized such that defining infarct/border zone and viable tissue becomes subjective. To optimize histological analysis, high-resolution histology and immunohistochemistry is used to guide the immunohistological interpretation.

Inflammatory response. Inflammation is graded semi-quantitatively on a 5-point scale (none, minimal, mild, moderate or severe) based on the density and type of inflammatory cells present. A similar scoring system is used to evaluate the degree of cell and tissue and infiltration into the grafts. Eosinophil response is measured by counting cells on a calibrated grid. Tissue thickness over grafts and in the surrounding tissue are measured with a calibrated stage. Left ventricular internal dimension at end systole (LVIDs) are measured by echocardiography (Khalpey Z, Penick K, Constantopoulos E, Garcia J, Sweitzer N, Runyan R, and Konhilas J. Meeting abstract submitted to American Heart Association. 2014).

Cellular and molecular analysis: A portion excised hearts is flash frozen (liquid nitrogen) for cellular and molecular interrogation. Total RNA and protein is isolated from the left ventricles and analyzed for expression of pathological markers of cardiac hypertrophy. Real time PCR is done via Universal ProbeLibrary Assay (Roche) using LightCycler 480 system (Roche); protein analysis is executed using SDS-PAGE and Western Blot analysis as previously described (Chau et al., supra; Konhilas J P, Watson P A, Maass A, Boucek D M, Horn T, Stauffer B L, Luckey S W, Rosenberg P, and Leinwand L A. Exercise can prevent and reverse the severity of hypertrophic cardiomyopathy. Circ Res. 2006; 98(4):540-8. 48. Konhilas J P, Chen H, Luczak E, McKee L A, Regan J, Watson P A, Stauffer B L, Khalpey Z I, McKinsey T A, Horn T, et al. Diet and sex modify exercise and cardiac adaptation in the mouse. Am J Physiol Heart Circ Physiol. 2015; 308(2):H135-45). Apart from known pathological indicators of cardiac remodeling, MI and PAM allografts may induce a unique pattern of pathological remodeling. Therefore, unbiased proteomic and metabolomics analysis is performed as described below. At each timepoint, blood from infarct and non-infarct groups is placed in lavender-top EDTA tubes, centrifuged to obtain plasma, and rapidly frozen in liquid nitrogen. This is the preferred method for determinations of circulating cytokines and chemokines as well as metabolomics analysis (de Jager W, Bourcier K, Rijkers G T, Prakken B J, and Seyfert-Margolis V. Prerequisites for cytokine measurements in clinical trials with multiplex immunoassays. BMC immunology. 2009; 10(52)).

Metabolomics

Recent metabolomics studies demonstrate the potential for implementing such as strategy for atrial fibrillation and new-onset atrial fibrillation (Alonso A, Yu B, Qureshi W T, Grams M E, Selvin E, Soliman E Z, Loehr L R, Chen L Y, Agarwal S K, Alexander D, et al. Metabolomics and Incidence of Atrial Fibrillation in African Americans: The Atherosclerosis Risk in Communities (ARIC) Study. PLoS One. 2015; 10(11):e0142610; Ko D, Riles E M, Marcos E G, Magnani J W, Lubitz S A, Lin H, Long M T, Schnabel R B, McManus D D, Ellinor P T, et al. Metabolomic Profiling in Relation to New-Onset Atrial Fibrillation (from the Framingham Heart Study). Am J Cardiol. 2016; 118(10):1493-6; Mayr M, Yusuf S, Weir G, Chung Y L, Mayr U, Yin X, Ladroue C, Madhu B, Roberts N, De Souza A, et al. Combined metabolomic and proteomic analysis of human atrial fibrillation. J Am Coll Cardiol. 2008; 51(5):585-94; Zhang Y, Blasco-Colmenares E, Harms A C, London B, Halder I, Singh M, Dudley S C, Gutmann R, Guallar E, Hankemeier T, et al. Serum amine-based metabolites and their association with outcomes in primary prevention implantable cardioverter-defibrillator patients. Europace. 2016; 18(9):1383-90). These studies also identify the limitations and pitfalls of such an approach such as inconsistent diagnosis, site specific sampling, and non-uniform metabolite coverage (Ko et al., supra). Furthermore, metabolomics quantitatively measures a multiparametric metabolic response of a dynamic cellular system. Consequently, sampling from a single timepoint captures only a snapshot of dynamic processes in response to a pathophysiological insult. The strategy described herein overcomes these limitations by executing a longitudinal examination of metabolites within each subject across a continuum pre-, peri- and postoperative. Factors that are either increased or decreased during CABG and triggering NOPAF are identified. Placement of PAM during CABG returns these pro-arrhythmogenic factors to preoperative levels. In addition, left atrial appendage is procured perioperatively and snap-frozen in liquid nitrogen for metabolomics and proteomic interrogation.

Proteomics and Metabolomics: Non-targeted metabolomics of cardiac tissue and plasma is performed by Metabolon (Durham, N.C.). Through an established database, metabolites are matched with signaling pathways of interest for follow up assessment (Guo L, Milburn M V, Ryals J A, Lonergan S C, Mitchell M W, Wulff J E, Alexander D C, Evans A M, Bridgewater B, Miller L, et al. Plasma metabolomic profiles enhance precision medicine for volunteers of normal health. Proc Natl Acad Sci USA. 2015; 112(35):E4901-10). Cardiac tissue (left atrial appendage) from experimental groups is used to perform mass spectrometry (liquid chromatography-electrospray ionization-tandem mass spectrometry [LC-ESI-MS/MS]) following in-gel digestion of experimental samples.

### Example 7

### Characterization of Endotoxin Level of PAM Allograft Used for Cardiac Surgical Procedures

Several samples of the PAM allografts described and used in at least Examples 1-3 above (i.e., commercially available as AMNIOBAND® Membrane from MTF of Edison, N.J.) were assayed for bacterial endotoxin level using a kinetic chromogenic Limulus Amebocyte Lysate (LAL) assay kit and reagents (commercially available from Charles River, located in Wilmington, Mass., U.S.A.), and guidance provided in the following the United States Department of Health and Human Services, Food and Drug Administration (“FDA”) report: Guidance for Industry Pyrogen and Endotoxins Testing: Questions and Answers, June 2012 Compliance, Content current as of Mar. 22, 2018 (additional contributing agencies and entities include Center for Drug Evaluation and Research (CDER), Center for Biologics Evaluation and Research (CBER), Center for Veterinary Medicine (CVM), Center for Devices and Radiological Health (CDRH), and Office of Regulatory Affairs (ORA)).

Subsequent to standard processing, the PAM allografts were obtained after completion of the final drying step (and in its final packaging) and assayed for LAL content, using the kit and reagents from Charles River, and scanned using the EndoScan-V software, version 5.5.5 sp1 or 5.1.2, in accordance with the directions provided therewith.

Generally, according to the instruction provided with the LAL assay, the following procedure was performed. Samples were extracted in 40 ml of LAL reagent water. The resulting extract was then plated on a 96 well plate. The sample was run in duplicate, as well as spiked in duplicate, using standards purchased from Charles River. A curve is also run on each plate using a serial dilution of standards purchased form Charles River. After all samples and standards were plated, Lysate (purchased form Charles River) was added to each well. The plate was then placed in a spectrophotometer and read using the above-identified software from Charles River.

According to the aforesaid guidance provided in the FDA report cited above, a passing test is one resulting in an Endotoxin value of <0.05046 EU/mL (endotoxin units per milliliter), corresponding to the stringent CSF (cerebral spinal fluid)-contacting limit. Thirty-seven (37) donor lots have been tested using this assay, and every lot had an endotoxin value of <0.5046 EU/ml.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the presently disclosed and contemplated invention.

1. A method for treating, reducing, or preventing cardiac disorder, disease or damage in a subject having a heart, which comprises the step of contacting the heart, a portion of the heart, a component of the heart, or a combination thereof, with at least one construct comprising one or more placenta-derived compositions.

2. The method of claim 1, wherein the cardiac disorder is one or more of: cardiac arrhythmia, scar tissue on the heart or a portion or component thereof, a wound or wound closure site on the heart or on a portion or component thereof.

3. The method of claim 1, wherein contacting the heart or a portion thereof comprises placing the at least one construct on a surface of the heart, a portion of the heart, a component of the heart, or a combination thereof.

4. The method of claim 1, wherein each of the one or more placenta-derived compositions comprises, independently, one or more placental components selected from amnion, chorion, umbilical cord, and combinations thereof.

5. The method of claim 4, wherein each of the one or more placental membrane components, independently, further comprise native endogenous cells, which are viable, not viable, or a combination thereof.

6. The method of claim 1, wherein each of the at least one construct is, independently of other constructs, in the configuration of a sheet, a disc, a piece, a fragment, a particulate, a powder, a three-dimensional shape, a coating, a layer, a film, an elongated element, or a combination thereof.

7. The method of claim 1, wherein contacting the heart or a portion thereof comprises injecting the at least one construct onto the surface of the heart, a portion thereof, a component thereof, or a combination thereof.

8. A method for preventing or reducing the risk new onset postoperative atrial fibrillation (NOPAF) in a subject having a heart which is being, or has been or will be, subjected to a surgical procedure, the method comprising: contacting the heart, a portion of the heart, a component of the heart, or a combination thereof, with at least one construct comprising one or more placenta-derived compositions, wherein the step of contacting is performed before, during, or after the surgical procedure, or a combination thereof.

9. The method of claim 8, wherein contacting the heart or a portion thereof comprises placing the at least one construct on a surface of the heart, a portion of the heart, a component of the heart, or a combination thereof.

10. The method of claim 8, wherein each of the at least one construct is, independently of other constructs, in the form of a sheet, a disc, a piece, a fragment, a particulate, a powder, a three-dimensional shape, a coating, a layer, a film, an elongated element, or a combination thereof.

11. The method of claim 8, wherein the at least one construct comprises two or more constructs and each construct is, independently, adjacent, overlapping, or not in contact, with one or more of the other constructs.

12. The method of claim 8, wherein each of the one or more placenta-derived compositions comprises, independently, one or more placental membrane components selected from amnion, chorion, and combinations thereof.

13. The method of claim 12, wherein the surgical procedure is a coronary artery bypass graft surgery (CABG), the at least one construct comprises one or more layered sheet constructs, each comprising at least one dehydrated amnion membrane sheet and at least one dehydrated chorion sheet arranged to form the respective layered sheet construct, and the step of contacting is performed during the surgical procedure.

14. The method of claim 13, wherein each of the one or more layered sheet constructs is positioned, during the CABG procedure, proximate the heart and pericardium in positions and orientations whereby, when the CABG procedure is completed, each of the one or more layered sheet constructs contact at least a portion of a surface of the heart, whereby NOPAF is prevented or risk of NOPAF is reduced.

15. The method of claim 14, wherein during the CABG procedure, the pericardium is manipulated to expose and permit access to anterior aspects of the pericardium and portions of the heart, which comprise a right atrium, an anterior left ventricle, and an inferior right ventricle, wherein the one or more layered sheet constructs comprise at least a first layered sheet construct and a second sheet construct, and wherein the step of contacting comprises:
positioning the first layered sheet construct to contact and cover at least a portion of the right atrium of the heart, and
positioning the second layered sheet construct to contact and cover the anterior left ventricle of the heart.

16. The method of claim 15, wherein the first layered sheet construct is positioned either by: placement in direct contact with and on the right atrium, or placement on an anterior aspect of the pericardium, at an edge thereof, with or without an affixing device, in an orientation which approximates placement over the right atrium when the pericardium is returned to a natural closed position prior to completion of the CABG procedure, and
wherein the second layered construct is positioned either by: placement in direct contact with and on the anterior left ventricle, or placement on an anterior aspect of the pericardium, at an edge thereof, with or without an affixing device, in an orientation which approximates placement over the anterior left ventricle when the pericardium is returned to a natural closed position prior to completion of the CABG procedure.

17. The method of claim 15, wherein the one or more layered sheet constructs further comprise a third layered sheet construct, and wherein the step of contacting further comprises:
positioning the third layered sheet construct to contact and cover at least a portion of the inferior right ventricle.

18. The method of claim 17, wherein the third layered construct is positioned in an orientation which approximates placement over the inferior right ventricle when the pericardium is returned to a natural closed position prior to completion of the CABG procedure, either by: placement proximate to and aligned with the base of the heart, with or without an affixing device, or placement on an anterior aspect of the pericardium, at an edge thereof, with or without an affixing device.

19. The method of claim 14, wherein each of the one or more layered sheet constructs has dimensions comprising a width and a length, each of which is from about 3 to about 8 centimeters and the one or more layered sheet constructs cover a total area of from at least about 30 to about 150 square centimeters (cm²) on the surface of the heart after the CABG procedure is completed.

20. The method of claim 19, wherein the one or more layered sheet constructs comprise at least a first layered sheet construct and a second layered sheet construct, each of which has dimensions comprising a width and a length, each of which is from about 5 to about 7 centimeters and the first and second layered sheet constructs collectively cover a total area of from at least about 50 to about 150 square centimeters (cm²) on the surface of the heart after the CABG procedure is completed.
